(19) United States
(12) Patent Application Publication
(10) Pub. No.: US 2022/0007622 A1
(43) Pub. Date: Jan. 13, 2022

(54) FACILITY FOR FARMING INSECTS, COMPRISING AN AUTOMATED SENSOR-EQUIPPED GUIDED VEHICLE

(71) Applicant: YNSECT, Évry-Courcouronnes Cedex (FR)

(72) Inventors: Thibault DU JONCHAY, CHEVRIÈRES (FR); Arturo ESCAROZ CETINA, EINDHOVEN (NL); Solene COMPARAT, FRANCONVILLE (FR); Fabrice BERRO, PARIS (FR)

(73) Assignee: YNSECT, Évry-Courcouronnes Cedex (FR)

(21) Appl. No.: 17/296,293

(22) PCT Filed: Dec. 9, 2019
(86) PCT No.: PCT/FR2019/052984

§ 371 Date: May 24, 2021

(30) Foreign Application Priority Data
Dec. 12, 2018 (FR) 1872760

(51) Int. Cl. A01K 67/033; A01K 29/00; B65G 1/04; B65G 1/137

(52) U.S. Cl. CPC A01K 67/033; B65G 1/137; B65G 1/0492; A01K 29/005

(57) ABSTRACT

The invention relates to a facility for farming insects, the facility comprising automated guided vehicles (AGV) configured to move containers within the facility. The automated guided vehicles (AGV) include at least one automated sensor-equipped guided vehicle (AGVI) which is provided with at least one sensor for measuring a parameter that is characteristic of a state of the facility. The invention thus developed enables monitoring of the state of a facility to be optimised in a facility for farming insects by taking advantage of the means used to transport the containers within the facility.

The present invention concerns the field of farms for rearing insects.

It is directed in particular to farms for rearing insects having a high degree of automation. It is directed for example particularly to growing farms in which insects are reared in suitable containers.

In the context of a farm for rearing insects, which will serve as main example for illustrating the present invention, the insects concerned by the invention are for example the Coleoptera, Diptera, Lepidoptera, Isoptera, Orthoptera, Hymenoptera, Blattoptera, Hemiptera, Heteroptera, Ephemeroptera and Mecoptera, preferably, Coleoptera, Diptera, Orthoptera, Lepidoptera.

The term “insect” is employed to designate any stage of development of the egg or egg pod to the adult insect, and the invention is more particularly directed to the rearing of insects from the larval stage to the adult insect.

Insect farming is experiencing something of a boom. The production of insects has many attractions, whether for the agro-industry, as certain species of edible insects are rich in proteins, or in other industrial fields. Typically, the exoskeleton of insects is constituted in large part by chitin, a known derivative of which is chitosan. The applications of chitin and/or chitosan are many: cosmetics (cosmetic composition), medical and pharmaceutical (pharmaceutical composition, treatment of burns, biomaterials, corneal dressings, surgical sutures), dietetic and dietary, technical (filtering agent, texturing agent, flocculating agent or adsorbant, in particular for water filtration or pollution control), etc. In fact, chitin and/or chitosan are biocompatible, biodegradable and non-toxic materials.

Document FR3034622 presents a farm adapted for the rearing of insects at an industrial scale. The rearing implements rearing containers (typically tubs) which are stackable, in one or more columns, to form basic rearing units. The basic rearing units are stored, and, when a rearing operation is to be carried out, the containers are brought, by automated systems such as automatons, to a station configured for carrying out the operation, grouped into basic rearing units or ungrouped singly.

The insects thus live in a zone in which they grow and develop between the rearing operations. It is thus important in this zone and more generally in the whole of the farm to maintain environmental conditions favorable to their health, their well-being and their fast growth.

Environmental conditions in particular refers to the environmental parameters which are the temperature of the air, the hygrometry, and the level of carbon dioxide (CO₂) present in the air.

The environmental conditions thus make it possible to judge a state of the farm, but other parameters characteristic of that state of the farm must be controlled to ensure good conditions for rearing. Among these characteristic parameters of the state of the farm there may in particular be mentioned the absence (or on the contrary the presence) of parasitic species in the farm.

Document CN107372375 indicates in general terms the importance of controlling the temperature, the humidity and the level of CO₂ in a silk worm farm. This document describes premises for rearing comprising a sensor of temperature, humidity in the air, and CO₂. However, simply verifying these parameters in an insect farm at an industrial scale proves to be insufficient in numerous respects. The overall verification proposed does not make it possible to detect a local anomaly or a deviation in an environmental parameter. Such a local event is nevertheless liable to affect part of the farm, and may also be the sign of an event liable to affect the whole of the farm. Furthermore, in the context of rearing at very large scale, it would be complex and costly to provide for covering the whole of the rearing farm with sensors.

The present invention is thus directed to a farm for rearing insects, in which the issues referred to below are solved in whole or in part.

In particular, the invention relates to a farm for rearing insects, said farm comprising a first zone in which the insects are stored during their growth in containers and a second zone comprising at least one station configured for the carrying out of an operation on the insects of a rearing container or on said container, the farm comprising automated guided vehicles configured for the movement of the rearing containers in the farm, that is to say within the first zone, and/or within the second zone, and between the first zone and the second zone. The automated guided vehicles comprise at least one instrument-equipped automated guided vehicle, said instrument-equipped automated guided vehicle being equipped with at least one sensor configured to perform a measurement of a parameter characteristic of a state of the farm.

The invention thus provides for taking advantage of the use in the farm of automated guided vehicles for the transport of growing or rearing containers to carry out a verification of a state of the farm by the measurement of at least one characteristic parameter. The solution thus provided has multiple advantages. A random verification may be carried out subject to the movements of the automated guided vehicle in its missions of transporting containers. A specific verification for example in a desired location of the farm, may be carried out simply and according to requirement by configured programming of the movement of an instrument-equipped automated guided vehicle.

The instrument-equipped automated guided vehicle may comprise, as sensor configured for measuring a parameter characteristic of a state of the farm, a humidity sensor, a temperature sensor, or a sensor of the carbon dioxide level. Furthermore or alternatively, the instrument-equipped automated guided vehicle comprises, as sensor configured to measure a parameter characteristic of a state of the farm, a movement sensor, an optical or thermal camera, or a sensor of resistance to movement.

The state of the farm is thus defined for example by one or more environmental parameters ambient in the farm at the location where a measurement is carried out, such as the temperature, the humidity of the air, or the CO2 level. However, other parameters may be taken into account. In particular, the presence of parasitic species (in particular parasitic insects) in the farm may be detected by virtue of direct parameters, that is to say their detection and their optical recognition or by indirect markers of their presence.

The sensor configured to measure a parameter characteristic of a state of the farm may be fixed in relation to a body of the instrument-equipped automated guided vehicle, or, alternatively, the instrument-equipped automated guided vehicle may be configured such that the sensor configured to measure a parameter characteristic of a state of the farm may be oriented or moved in relation to the body of the instrument-equipped automated guided vehicle.

The instrument-equipped automated guided vehicle may comprise a device for wireless transmission configured to emit a signal corresponding to the measurement of the characteristic parameter.

The instrument-equipped automated guided vehicle may comprise a computer memory in which the characteristic parameter is stored and a communication port enabling said memory to be unloaded by a wired or wireless channel.

A wireless transmission device enables real-time detection of an anomaly in the state of the farm. Additional verifications or corrective measures may then be implemented. A system enabling the storage of the measurements in a memory makes it possible to defer their transmission or ensure that a copy remains accessible in case of reception failure. In general, for farms of large size or for economic or technical reasons, it may be preferable to provide for transmission of the signals corresponding to the measurements made that is only periodic.

The farm may comprise a central system for verifying the state of the farm configured to receive from the instrument-equipped automated guided vehicle (IAGV) a representation signal representing the measurement of the characteristic parameter and to determine, according to said representation signal correlated with an information item on position of the instrument-equipped automated guided vehicle at the time of said measurement, the presence of a local anomaly in the state of the farm.

Thus, the verification of the state of the farm carried out conventionally by fixed sensors may be complemented with the measurements made by the instrument-equipped automated guided vehicle. By complemented is meant producing equivalent measurements for the purpose of a comparison (for example to confirm an anomaly detected by a fixed sensor), producing measurements in case of a fault of a fixed sensor, producing more precise measurements (for example more localized) or producing measurements in the zones of the farm which are not covered by the fixed sensors.

The farm may comprise a central system for verifying the state of the farm configured to receive from the instrument-equipped automated guided vehicle a representation signal representing the measurement of the characteristic parameter and to determine, according to said representation signal correlated with an information item on position of the instrument-equipped automated guided vehicle at the time of said measurement, the presence of a local anomaly in the state of the farm.

The central system for verifying the state of the farm may furthermore be adapted to receive from the fixed sensors representation signals representing measurements of the characteristic parameter and to determine the presence of a local anomaly in the state of the farm according to the representation signals, each representation signal being correlated with an item of information on identity of the fixed sensor which emits it.

The farm may comprise a central guidance system, said central guidance system being configured to transmit guidance information to said automated guided vehicles in particular for example to said instrument-equipped automated guided vehicle.

The central guidance system may be configured to transmit to the instrument-equipped automated guided vehicle guidance information in response to a local anomaly determined on the basis of a signal emitted by one of the fixed sensors.

The central guidance system may be configured to perform periodic transmission to the instrument-equipped automated guided vehicle of guidance information in order to make it inspect part of the farm which is not covered by a fixed sensor of the farm to measure the characteristic parameter there.

The central guidance system may be configured to perform periodic transmission to the instrument-equipped automated guided vehicle, of guidance information in order to make it inspect part of the farm in which the automated guided vehicles do not pass when transporting containers.

The verification and the management of the state of the farm may thus be envisioned globally. In particular, the central system for verifying the state of the farm may be interfaced with the system controlling the movements of the automated guided vehicles. The movements of the instrument-equipped automated guided vehicle or vehicles may thus be managed to perform optimally both their transport missions and their missions for verifying the parameters to monitor.

The invention also relates to a method for verifying a state of a farm for rearing insects comprising a first zone in which the insects are stored during their growth in containers and a second zone comprising at least one station configured for the carrying out of an operation on the insects of a container or on said container, the farm comprising automated guided vehicles configured for the movement of the containers in the farm, that is to say within the first zone and/or within the second zone, and between the first zone and the second zone.

said method being characterized by the measurement of a parameter characteristic of the state of the farm by an instrument-equipped automated guided vehicle equipped with at least one sensor configured to make the measurement of said parameter characteristic of the state of the farm.

Still other particularities and advantages of the invention will appear in the following description.

In the accompanying drawings, given by way of non-limiting example:

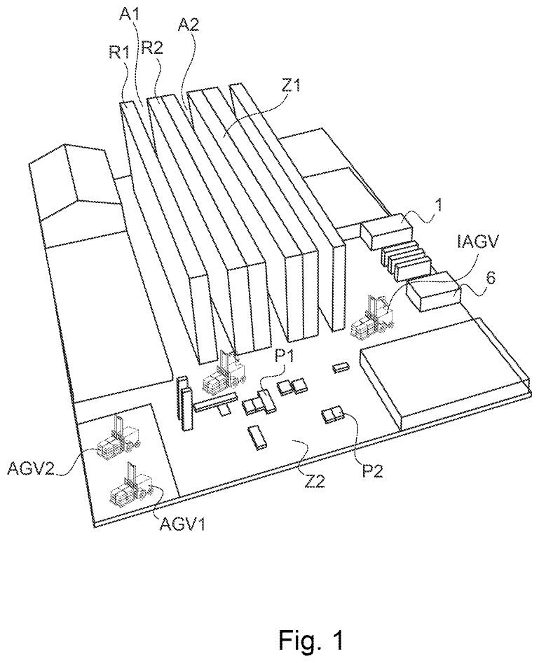

FIG. 1 shows, in a three-dimensional diagrammatic view, an insect rearing farm example in accordance with one embodiment of the invention;

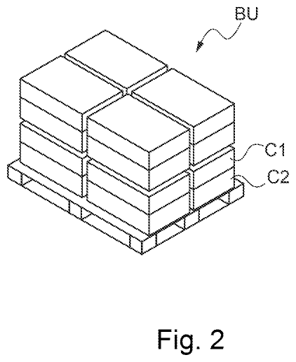

FIG. 2 shows a diagrammatic view in three dimensions of a set of rearing containers which can be used in an insect farm;

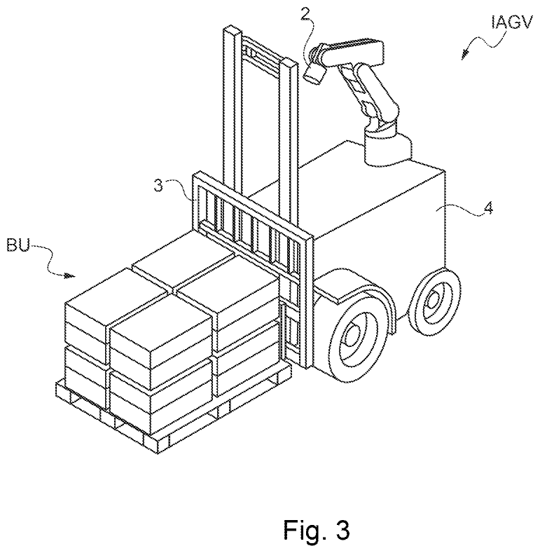

FIG. 3 shows a first example of an automated guided vehicle able to be implemented in the invention;

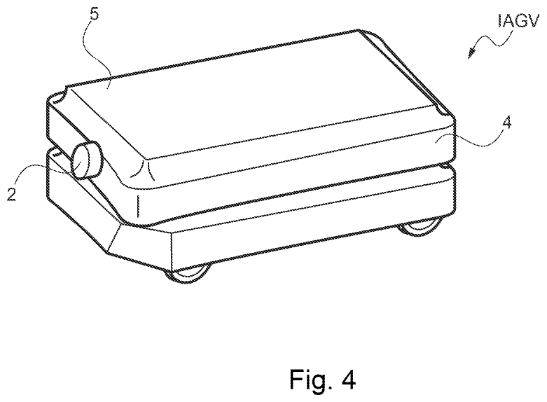

FIG. 4 shows a second example of an automated guided vehicle able to be implemented in the invention;

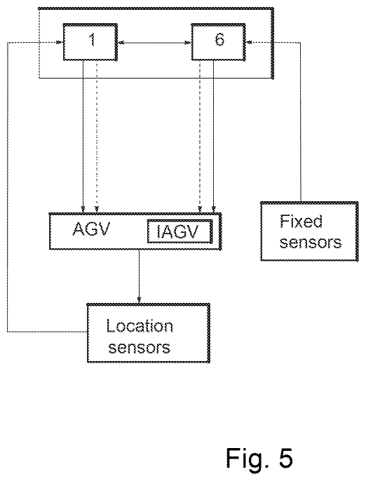

FIG. 5 shows, in a concept diagram, an example of the data streams which may be established in a farm in accordance with an embodiment of the invention;

FIG. 1 shows an insect rearing farm, here represented in the form of a diagrammatic view in three dimensions.

The rearing of insects can in particular be envisioned as an organized facility enabling the laying of eggs by adult insects for the production of larvae, some larvae being reared to reach the adult stage for the laying of new eggs, the adults being renewed regularly (for example further to their death) by young adults ensuring new laying and so forth. The final product of the production can be eggs, and/or larvae, and/or nymphs, and/or adult insects.

As shown in FIG. 1, a farm for rearing insects according to the invention, shown here diagrammatically in three dimensions, comprises at least two zones, namely a first zone Z1 organized for the storage of the insects during their growth. In this first zone Z1, the insects increase in size under controlled and optimized environmental conditions (defined by environmental parameters including temperature, hygrometry, etc.).

The farm also comprises a second zone Z2, organized for carrying out of one or more rearing operations. The rearing operations correspond to operations that must be conducted in order to maintain life, good growth and/or the optimization of the insect rearing conditions.

The harvesting of the insects constitutes an operation that can be conducted in the second zone Z2.

The second zone Z2 in particular comprises, in the example shown in FIG. 1, one or more work stations P1, P2, specialized for carrying out one or more rearing operations.

The insects (eggs, larvae, nymphs, or adults) are reared in containers, which may be grouped into sets called basic rearing units. In growth phases, the containers are stored in the storage zone Z1, for example in racks R1, R2 for pallets.

An example of a basic rearing unit is shown in FIG. 2 in a representation of principal in three dimensions. In order to facilitate the handling thereof, each basic rearing unit may be carried by a pallet, as shown in FIG. 2.

In particular, the rearing containers C1, C2 can be stackable crates or tubs. By stackable tubs or crates is meant in particular tubs or crates that are superimposed on one another in a slightly nested manner, which achieves a certain stability for the column of crates thus formed.

As shown in FIG. 2, the containers C1, C2 are palletized, i.e. grouped together into basic units BU on a loading pallet 3. The pallet may particularly, but not exclusively, be a pallet of conventional size, i.e. typically a “pallet Europe” type of pallet, or a half-pallet of that type.

By way of example, a basic rearing unit BU can typically group together eight to one hundred containers C1, C2, and comprise one, two, three, or four piles of containers, or even more. The height of an entire basic rearing unit may for example be comprised between 160 and 230 cm, and typically of the order of 200 cm.

In the example of organization shown in FIG. 1, the racks of pallets are separated by parallel aisles A1, A2 enabling passage between the racks R1, R2. The rearing containers, for example in the form of crates that are single or grouped into basic units, must be transported within the farm several times during rearing. In particular, the containers must be recovered in the first zone Z1 to be transported into the second zone Z2 where operations are carried out. Similarly, the containers coming from the second zone Z2 must generally be transported into the first zone Z1 to be stored there, for example in racks for pallets or in similar structures.

Moreover, transport of the containers, whether or not in palletized form, may be necessary within the first zone Z1, or between different stations of the second zone Z2.

For this, the farm comprises automated guided vehicles AGV1, AGV2. The automated guided vehicles or autonomous vehicles are often designated by the acronym AGV. In an industrial context, they are automatons able to move in accordance with guidance information which may be supplied to said automated guided vehicles AGV1, AGV2 by various means. The vehicle may implement various automation technologies for its guidance, which may be implemented by wired guidance, vision guidance, and/by more flexible technologies based on geolocation and geoguidance of the vehicle in the farm. As the case may be, one or more cameras or ultrasound systems enable the vehicle to perceive its spatial environment and adapt its movements according to the latter.

The automated guided vehicles implemented may be of various types. In the context of a farm employing basic units in the form of sets of palletized containers, the vehicles may advantageously have the shape of an automated pallet jack, such as represented and described in more detail hereinafter with reference to FIG. 3.

Simpler automated vehicles may be employed, for example for the transport of containers that are single or in the form of a simple pile of crates. Such automated guided vehicles are illustrated and described below with reference to FIG. 4.

The automated guided vehicles implemented in the invention have first of all the role of ensuring the flow of containers in the insect rearing farm; that is to say of transporting the containers according to requirement. To that end, a central guidance device 1 may be provided. The central guidance device 1 comprises information technology means required for planning the movements of each automated guided vehicle, and of transmitting to said vehicle the information required for its guidance. This information may consist of a complete path which a given automated guided vehicle must follow, or, if technologies such as wire guidance or vision guidance are used, this information may consist of a command to follow a given track. The information also contains the instruction for the containers to retrieve, as well as their destination.

Thus, the central guidance system 1 makes it possible to attribute transport missions individually to the automated guided vehicles AGV1, AGV2.

The transmission of the guidance information is advantageously carried out by wireless means. The automated guided vehicle AGV1, AGV2 may also send information in return to the central guidance device 1. In particular, in the case of a geolocated vehicle in the farm, the central guidance device may receive information from location sensors present in the farm but also from the automated guided vehicle itself. If the automated guided vehicle is equipped with a camera or an ultrasound device, or laser device, for its guidance, the data provided by that device may be transmitted to the central guidance device which will be able to send new guidance information in reply. Thus, a large part of the software component, or even of the intelligence, necessary for the guidance of the automated guided vehicles may be centralized at the central guidance device.

A farm in accordance with the present invention comprises among the automated guided vehicles at least one instrument-equipped automated guided vehicle IAGV.

The automated guided vehicle is said to be instrument-equipped in that it comprises a sensor adapted to perform a measurement of a parameter characteristic of a state of the farm.

By state of the farm, reference is made to certain criteria and conditions that are propitious or on the contrary inappropriate for the rearing of the insects reared in the farm.

By parameter characteristic of a state of the farm, reference is thus made in particular to the environmental parameters able to be monitored conventionally, that is to say the temperature, the humidity of the air, and the carbon dioxide content. These parameters directly express certain conditions that are propitious or inappropriate to the life of the insects of the farm, or to their growth. They may also characterize conditions favorable to the appearance of mold, fungus, and fungal diseases of the insects reared.

However, other criteria may be controlled on the basis of the environmental parameters or other characteristic parameters. In particular, the absence of certain pollutants in the air of the farm (carbon monoxide, nitrogen oxide, ammonia, methane, volatile sulfur-containing products such as hydrogen sulfide) may be verified using a suitable sensor. Similarly, the presence of certain spores in the air may be detected using a configured sensor, and more generally detection of the microbial content may be provided.

Similarly, illumination may be an important parameter for some farming.

Furthermore, among the features of a state of the farm, it is important to verify the presence, or the absence, of parasitic species in the farm. In particular, certain diptera or arachnids may develop in the farm. These parasitic species, not desired in the farm, may be vectors of diseases for the insects of the farm, or become competitors thereto at least in certain zones of the farm.

Such species may be detected directly, for example using a camera. A species may be detected by image recognition. Certain species may be detected even in the absence of image recognition, for example by detecting the path of an object in the images coming from the camera. In this connection, it is notable that a same camera may be employed for guiding the automated guided vehicle and for detecting a parameter characteristic of the state of the farm, for example the presence of a parasitic species. However, in this case the instrument-equipped vehicle with automatized guidance is distinguished from the conventional automated guided vehicles in that its sensor is configured, in the processing of the data coming therefrom, for measuring the presence or the absence of a parasitic species. A dedicated camera may also be employed, whether or not the automated guided vehicle is equipped with a camera for its guidance.

Alternatively or in a complementary manner, a movement sensor equipping the instrument-equipped automated guided vehicle may be employed for detecting the presence of a parasitic species.

Moreover, such species may be detected by indirect markers of their presence. One of these markers may be the presence of silk or webs, for example mechanically detected by the resistance to movement they cause. The instrument-equipped automated guided vehicle may to that end comprise a force sensor making it possible to detect a resistance to movement (for example to the advancement of the vehicle, or a resistance on movement of a movable arm comprised by the instrument-equipped automated guided vehicle). An optical detection of silk or webs may also be employed.

Another indirect marker may be the heat caused by the movement similar to Brownian motion of these parasitic species. Resulting from this is a point, or a small zone, of which the temperature is greater than that of its immediate environment. A temperature sensor making it possible to measure a temperature locally enables this characterization, and likewise a thermal camera.

FIG. 3 shows an automated guided vehicle that can be employed in a farm in accordance with one embodiment of the invention. The automated guided vehicle shown is in particular an instrument-equipped automated guided vehicle IAGV in that it is equipped with a sensor 2.

The automated guided vehicle shown is of automated guided pallet jack type. This vehicle is particularly suited to the transport of pallets, in that it comprises a fork configured for lifting and holding a conventional pallet. This vehicle is also configured, by virtue of a lifting system 3, to go to retrieve pallets in the top part of the pallet racks R1, R2.

In FIG. 3, the automated guided vehicle is shown bearing a palletized basic unit BU.

The sensor 2 may be of any type mentioned above. It may for example be a thermal camera. The sensor 2 is movable in relation to the body 4 or chassis of the automated guided vehicle. To that end, in the example shown, the sensor is mounted on a robot arm movable on several axes. This type of mounting gives rotational and translational mobility to the sensor 2. The sensor 2 may thus be oriented towards a particular point of interest, or be brought away or towards such a point of interest.

Other systems making it possible to make the sensor mobile may be envisioned in the context of the invention: a sensor mounted on a simple swivel, telescopic arm whether or not swiveled, etc.

Several sensors may equip a same instrument-equipped automated guided vehicle IAGV. The sensors may in this case be mounted by a single same system providing their mobility, or on different systems. Certain sensors may be mobile, others fixed.

In FIG. 4, the automated guided vehicle shown comprises a fixed sensor 2. Fixed is to be understood in relation to the body 4 of the vehicle. The automated guided vehicle shown in FIG. 4 is of a flatbed dolly type 5. The flatbed dolly 5 enables a load to be transported to be deposited thereon. This load may be a container (for rearing or growth), a stack of containers, a basic unit which may be palletized as may be appropriate, although in the context of a farm employing palletized groups, a more advantageous type of automated guided vehicle may be chosen.

Of course, the automated guided vehicle of the type shown in FIG. 4 may be equipped with one or more mobile sensors, for example rendered mobile using one of the systems referred to above, alternatively or in addition to one or more fixed sensors.

For example, in the embodiment of FIG. 4, the sensor 2 may be a camera, having a function of correlation of the guidance of the automated guided vehicle (for example for the avoidance of an obstacle) and moreover being configured to measure a parameter characteristic of a state of the farm, for example for detecting the presence of flying or crawling parasitic insects.

The sensors equipping the instrument-equipped automated guided vehicle or vehicles which the farm comprises (all the automated guided vehicles of the farm may be instrument-equipped, or only some of those vehicles) makes it possible to make measurements at will of the movements of the instrument-equipped automated guided vehicles in the context of the transport of containers, and/or according to requirement when making trips in the farm having as main objective to make such measurements.

The farm, for example the farm shown in FIG. 1, may comprise fixed sensors for monitoring the state of the farm. Generally, the farm may be equipped with sensors of the environmental conditions which are temperature, humidity level, and/or the level of carbon dioxide in the air. The measurements made by the automated guided vehicles make it possible in this case to complement the measurements made by the fixed sensors of the farm. “Complement” is to indicate that this may be to measure, using an instrument-equipped automated guided vehicle, a same parameter as that measured by a fixed sensor of the farm, or a parameter closely linked to the latter, and to check the consistency of the measurements respectively made by the fixed sensor of the farm and the sensor of the instrument-equipped automated guided vehicle. It may also be to spatially complement the measurements of the fixed sensors of the farm, for example by measuring the parameter characteristic of part of the farm which is not covered by the fixed sensors. Lastly, it may be to qualitatively complement the measurements by the fixed sensors of the farm. In other words, it may also be to produce measurements of a parameter which is not measured by the fixed sensors. For example the fixed sensors of the farm may measure the temperature, the humidity level, and the CO₂ level, while the instrument-equipped automated guided vehicle makes it possible to detect for example the presence of a parasitic species, or to detect the development of a fungus in the farm (whether this is in the containers or not, for example on the walls of the farm).

To ensure an effective verification of one or more characteristic parameters of the state of the farm, the farm advantageously comprises a central system 6 for verifying the state of the farm. The central verifying system comprises a set of electronic or information technology systems configured to collect, for example in the form of representation signals, the measurements made by the automated guided vehicles, and by the fixed sensors of the farm when the latter comprises these. The measurements are processed by the central verifying system for example in order to detect a local anomaly, which may indicate a particular or feared event. The anomaly may consist in measuring a value of the measured parameter outside an acceptable value range, or in directly detecting an event (movement, warm point, presence of silk or web indicating the presence of a parasitic species in the farm).

The central system 6 for verifying the state of the farm is advantageously interfaced with the central guidance system. The interfacing between the central system for verifying the state of the farm and the central guidance system consists in enabling an exchange of information between these systems, for example such that information on the position and the route of each automated guided vehicle, whether or not instrument-equipped, may be taken into consideration by the central system for verifying the state of the farm for controlling the measurements for the purpose of verifying the state of the farm. Indeed, one of the advantages of employing instrument-equipped automated guided vehicles is to make a measurement according to need. In particular, an instrument-equipped automated guided vehicle may be sent to a given location of the farm where a measurement coming from a fixed sensor of the farm is considered abnormal. Furthermore, an instrument-equipped automated guided vehicle may be sent periodically to take the measurement of the parameter (or parameters) characteristic of a part of the farm which is not covered by a fixed sensor. Furthermore, an instrument-equipped automated guided vehicle may be sent periodically to make the measurement of the parameter (or parameters) characteristic of part of the farm which is never or only rarely on the path of the automated guided vehicles of the farm as part of their missions of container transport. Lastly, when the central system for verifying the state of the farm finds, by virtue of the information coming from the central guidance system, that no vehicle with instrument-equipped guidance has gone to a given part of the farm in the course of its missions of rearing container transport, it may decide to send one there, in the context of a container transport mission or if needed specifically to perform a verification.

The transmission of the data from each instrument-equipped automated guided vehicle IAGV to the central system for verifying of the state of the farm 6 (and according to requirement from the central verifying system to an instrument-equipped automated guided vehicle) is preferentially carried out by wireless communication means. The wireless communication means envisioned include for example particularly communication systems implementing at least one of the following protocols: NFC, RFID, Bluetooth, and in particular Bluetooth Low Energy, Lifi, and Zigbee (registered trademarks). Other protocols may of course be employed.

According to the needs of the protocol employed, the communications between instrument-equipped automated guided vehicle IAGV and the central verifying system 6 may be made continuously or only periodically.

By “continuously” it is in particular envisioned that the measurements be transmitted instantaneously to the central system for verifying of the state of the farm 6, for example in step with the frequency of acquisition of the sensor or sensors equipping the vehicle.

“Periodically” corresponds to a discontinuous transmission, for example based on a given interval of time, or when the automated guided vehicle passes a certain point, in range of transmission of the central system for verifying of the farm or of a reception relay, or for instance when it is at a fixed station for example when the automated guided vehicle is electric with batteries and is in course of being charged.

It may also be provided, for a periodic transmission, to transmit the data to the central system for verifying the state of the farm by wired channel, via a physical port of the instrument-equipped automated guided vehicle. A wired communication can also enable the programming of the instrument-equipped automated guided vehicle, as regards its guidance or the measurements it is to make, in particular when the central guidance system and the central system for verifying of the state of the farm are interfaced.

FIG. 5 illustrates an example of the data streams which may be implemented in an exemplary farm. For this example, an insect rearing farm is considered comprising automated guided vehicles AGV some of which are instrument-equipped. The automated guided vehicles may be located by location sensors present in the farm. The farm comprises a central guidance system 1 and a central system 6 for verifying the state of the farm. Fixed sensors are disposed in the farm.

The central guidance system 1 and the central system 6 for verifying the state of the farm are interfaced such that the central guidance system 1 supplies the central system 6 for verifying of the state of the farm with data relative to the position of the instrument-equipped automated guided vehicles in the farm, as well as relative to the routes to which they are attributed. The central system 6 for verifying the state of the farm may send the central guidance system 1 instructions in order for the central guidance system to attribute a given route, or a route passing within a given part of the farm, to an instrument-equipped automated guided vehicle. Such an attribution may for example be ordered when a fixed sensor of the farm sends the central system 6 for verifying a state of the farm an abnormal measurement, or to make the measurement of a parameter in part of the farm in which a measurement must be made periodically, whether or not that part of the farm is covered by fixed sensors, and whether or not that part of the farm is a part in which automated guided vehicles pass in the context of the mission of container transport.

Thus, the central guidance system 1 individually sends the automated guided vehicles AGV, and in particular the instrument-equipped automated guided vehicles IAGV, the data necessary for their movements in the farm. In return, the automated guided vehicles, whether or not instrument-equipped, may according to requirement send data relative to their movement to the central guidance system 1. These data may for example be information on their movement, in order to enable their location or confirm their location, or information on the performance of certain tasks as steps within the route attributed to given a automated guided vehicle. These data may also be an output of information in real-time making it possible to adapt the guidance of the vehicles, for example a video output.

For the location in the farm of the instrument-equipped automated guided vehicles, it is provided in the example represented here for the farm to comprise a set of location sensors. They may be sensors enabling reception of a position signal emitted by the automated guided vehicles, for example an RFID signal (RFID standing for radio frequency identification), or sensors simply making it possible to detect the passage or the presence of an automated guided vehicle and to inform the central guidance system thereof, which may correlate this information with the routes attributed to each automated guided vehicle.

The instrument-equipped automated guided vehicles IAGV are, for their part, configured to transmit the measurements they make to the central system 6 for verifying the state of the farm. This transmission may be carried out based on various procedures and various protocols referred to above.

In return the central system 6 for verifying the state of the farm may possibly send information relative to the measurements to make, either directly, or via the guidance information coming from the interfaced central guidance system 1.

FIG. 5 is of course a simple example of the data streams that may be implemented in an insect rearing farm in accordance with one embodiment of the invention. Other configurations are possible, the insect rearing farm taken as a basis for this example comprising several optional devices in the context of the invention.

The invention thus set forth enables the optimization of the verification of the state of an insect rearing farm, by taking advantage of the means used for the transport of the containers in the farm. In particular, by equipping one or more automated guided vehicles with instruments, high flexibility in the verifications may be obtained, as well as, if required, coverage of the whole of the farm. Furthermore, the use of systems provided in the farm for the function of transportation makes it possible to obtain a function of verification of the state of the farm at a lower cost than the use of systems entirely dedicated to verification.

1. A farm for rearing insects, said farm comprising a first zone (Z1) in which insects are stored during their growth in containers (C1, C2) and a second zone (Z2) comprising at least one station (P1, P2) configured for performing an operation on the insects of a container or on said container;
the farm comprising automated guided vehicles (AGV) configured for the movement of the containers in the farm, that is to say within the first zone (Z1), and/or within the second zone (Z2), and between the first zone (Z1) and the second zone (Z2),
characterized in that said automated guided vehicles (AGV) comprise at least one instrument-equipped automated guided vehicle (IAGV), said instrument-equipped automated guided vehicle (IAGV) being equipped with at least one sensor (2) configured to perform a measurement of a parameter characteristic of a state of the farm.

2. The farm for rearing insects according to claim 1, wherein the instrument-equipped automated guided vehicle (IAGV) comprises, as sensor (2) configured for measuring a parameter characteristic of a state of the farm, a humidity sensor, a temperature sensor, or a sensor of the carbon dioxide level.

3. The farm for rearing insects according to claim 1, wherein the instrument-equipped automated guided (IAGV) vehicle comprises, as sensor (2) configured to measure a parameter characteristic of a state of the farm, a movement sensor, an optical or thermal camera, or a sensor of resistance to movement.

4. The farm for rearing insects according to claim 1, wherein the sensor (2) configured for measuring a parameter characteristic of a state of the farm is fixed relative to a body (4) of the instrument-equipped automated guided vehicle (IAGV).

5. The farm for rearing insects according to claim 1, wherein the sensor (3) configured for measuring a parameter characteristic of a state of the farm may be oriented or moved relative to a body (4) of the instrument-equipped automated guided vehicle (IAGV).

6. The farm for rearing insects according to claim 1, wherein the instrument-equipped automated guided vehicle (IAGV) comprises a device for wireless transmission configured to emit a signal corresponding to the measurement of the characteristic parameter.

7. The farm for rearing insects according to claim 1, wherein the instrument-equipped automated guided vehicle (IAGV) comprises a computer memory in which the characteristic parameter is stored and a communication port enabling said memory to be unloaded by a wired or wireless channel.

8. The farm for rearing insects according to claim 1, further comprising fixed sensors for measuring the parameter characteristic of a state of the farm.

9. The farm for rearing insects according to claim 1, comprising a central system (6) for verifying the state of the farm configured to receive from the instrument-equipped automated guided vehicle (IAGV) a representation signal representing the measurement of the characteristic parameter and to determine, according to said representation signal correlated with an information item on position of the instrument-equipped automated guided vehicle at the time of said measurement, the presence of a local anomaly in the state of the farm.

10. The farm for rearing insects according to claim 8, wherein the central system (6) for verifying the state of the farm is furthermore adapted to receive from the fixed sensors representation signals representing measurements of the characteristic parameter and to determine the presence of a local anomaly in the state of the farm according to the representation signals, each representation signal being correlated with an item of information on identity of the fixed sensor which emits it.

11. The farm for rearing insects according to claim 9, comprising a central guidance system (1), said central guidance system (1) being configured to transmit guidance information to said automated guided vehicles (AGV) in particular for example to said instrument-equipped automated guided vehicle.

12. The farm for rearing insects according to claim 10, wherein said central guidance system (1) is configured to transmit to the instrument-equipped automated guided vehicle (IAGV) guidance information in response to a local anomaly determined on the basis of a signal emitted by one of the fixed sensors.

13. The farm for rearing insects according to claim 10, wherein said central guidance system (1) is configured to perform periodic transmission to the instrument-equipped automated guided vehicle (IAGV) of guidance information in order to make it inspect part of the farm which is not covered by a fixed sensor of the farm to measure the characteristic parameter there.

14. The farm for rearing insects according to claim 11, wherein said central guidance system (1) is configured to perform periodic transmission to the instrument-equipped automated guided vehicle (IAGV), of guidance information in order to make it inspect part of the farm in which the automated guided vehicles do not pass when transporting containers (C1, C2).

15. A method Method for verifying a state of a farm for rearing insects comprising a first zone (Z1) in which the insects are stored during their growth in containers (C1, C2) and a second zone (Z2) comprising at least one station (P1, P2) configured for the carrying out of an operation on the insects of a container or on said container, the farm comprising automated guided vehicles (AGV) configured for the movement of the containers in the farm, that is to say within the first zone (Z1), and/or within the second zone (Z2), and between the first zone (Z1) and the second zone (Z2),
said method being characterized by the measurement of a parameter characteristic of the state of the farm by an instrument-equipped automated guided vehicle (IAGV) equipped with at least one sensor configured to make the measurement of said parameter characteristic of the state of the farm.
